(19) United States
(12) Patent Application Publication
(10) Pub. No.: US 2022/0378820 A1
(43) Pub. Date: Dec. 1, 2022

(54) GOLD NANOCLUSTER IN THE TREATMENT OF FRIEDREICH'S ATAXIA

(71) Applicant: NOVYSTEM S.P.A., MILANO (IT)

(72) Inventors: Angelo Maria MONGUZZI, Milano (IT); Chiara VILLA, Milano (IT); Yvan TORRENTE, Milano (IT)

(21) Appl. No.: 17/755,814

(22) PCT Filed: Nov. 10, 2020
(86) PCT No.: PCT/IB2020/060579

§ 371 Date: May 9, 2022

(30) Foreign Application Priority Data
Nov. 11, 2019 (IT) 102019000020724

(51) Int. Cl. A61K 33/242; A61P 25/16; A61P 25/28

(52) U.S. Cl. CPC A61K 33/242; A61P 25/16; A61P 25/28

(57) ABSTRACT

The present invention relates to superstructured gold clusters Au-pX, consisting of gold atoms and at least one ligand, where said gold atoms in said cluster are in a number between 2 and 100 or the dimensions of said cluster are smaller than 2 nm, for use in the treatment of pathologies related to oxidative stress. In a preferred embodiment, said Au-pX superstructured gold clusters are for use in the treatment of Friedreich's ataxia.

Friedreich's ataxia (FRDA) is a neurodegenerative disease caused by the unstable expansion of a GAA triplet located in intron 1 of the FXN gene (9q21.11), which encodes Frataxin. The disease is mostly characterized by progressive ataxia of gait and limbs, dysarthria, dysphagia, oculomotor dysfunction, loss of deep tendon reflexes, pyramidal signs, scoliosis and, in some cases, cardiomyopathy, diabetes mellitus, loss of vision and hearing impairment.

There is no resolutive therapy for FRDA, the symptomatological framework is now treated in a multidisciplinary way, typically with the support of physiotherapy, pharmacological agents such as baclofen and botulinum toxin for spastic control, antiarrhythmics and anticoagulants for cardiomyopathy.

LIM F, et al. Mol Ther. 2007; 15: 1072-1078; Vyas P M, et al. HUM mol Genet. 2012; 21: 1230-47; Jones J, et al. Mol Ther. 2015; 23: 130-138; Perdomini M, et al. Mean NAT. 2014; 20: 542-547 describe preclinical therapies with stem cells or gene therapies, tested to increase levels of healthy Frataxin.

Huntington's Disease (HD) is a neurodegenerative disease caused by the unstable expansion of a CAG triplet in the gene coding for huntingtin, on the short arm of chromosome 4 (4p16.3). The disease affects muscle coordination and leads to cognitive decline and psychiatric problems. To date, the available pharmacological treatments cannot alleviate many of the numerous symptoms.

Alzheimer's Disease (AD) is the most common form of progressively disabling degenerative dementia with predominantly presenile onset. Resolutive therapies are currently not available for Alzheimer's Disease.

Parkinson's Disease (PD) is a neurodegenerative disease. The motor symptoms typical of the condition are the result of the death of the cells synthesizing and releasing dopamine. A cure for Parkinson's Disease is not available to date, although pharmacological treatment, surgery and multidisciplinary management are able to provide relief to symptoms.

Oxidative stress is an important component of the pathogenesis of FRDA and can explain DNA damage and neuronal degeneration (Yokota T, et al. Proc Natl Acad Sci USA 2001; 98: 15185-15190). Oxidative stress has also been shown to be involved in the etiopathogenesis of HD (Kumara A, Ratana R R, J Huntington's Dis. 2016; 5(3): 217-237), AD (Markesbery W R, Free radical Biology and Medicine 1997; 23(1): 134-147) and PD (Henchcliffe C, Beal M F, Nature clinical practice 2008; 4(11): 600-609).

Clinical studies with antioxidants, such as Idebenone, MitoQ, CoQ10 and vitamin E, to reduce cellular oxidative damage have however had limited success.

Santiago-Gonzales B et al. Science 2016; 353-571-575 disclose stable superstructures formed by aggregates of gold atoms, called Au-pXs.

The present invention relates to a method for treating pathologies caused by an excess of oxygen radicals, in particular FRDA, which exceeds the limits of the therapies available to date.

### DESCRIPTION OF THE DRAWINGS

FIG. 1: Cyclic voltammogram of the PBS electrolyte before (solid line) and after (dashed line) the addition of an aqueous solution of H₂O₂ in the absence (A) or in the presence (B) of clusters Au₈-pX.

FIG. 2: Impact of the Au₈-pX clusters on the concentration of ¹O₂.

FIG. 3: Impact of the Au₈-pX clusters on the H₂O₂ concentration.

FIG. 4: Impact of the Au₈-pX clusters on the concentration of O.

FIG. 5: Neuromotor function in the YG8R mice injected Au₈-pX. (A) picture representative of the posterior and anterior extremity footprint test of YG8sR-PX treated Au₈ mice. (B) the footprint test showed an increase in pitch length (stride length) for Au₈-pX injected mice as compared to control animals (CTR). (C) the treadmill test (Treadmill Test) revealed an increase in exercise resistance, measured in number of pulses (number of shocks) for the injected mice Au₈-pX compared to the control. The values represent the mean ±SEM. P <0.05; ** P<0.01; P<0.001; ns=not significant. CTR=control animals.

FIG. 6: Cardiac function in YG8R mice injected Au₈-pX. (A) representative M-mode echocardiographic image of a mouse treated with Au₈-pX (injected) and untreated (untreated). (B) echocardiographic analysis revealed an increase in ejection fraction (EF) and fractional shortening (FS) in Au₈-pX injected mice relative to control. The values represent the mean±SEM. * P<0.05.

FIG. 7: Levels of antioxidant expression in dorsal root ganglia, cortex, cerebellum and basal ganglia. QRT-PCR analysis revealed a statistically significant superior expression of Prdx2, Gstm1 and Nrf2 in the dorsal root ganglia of the Au₈-pX injected mice compared to control. The data were normalized to GAPDH. The values represent the mean±SEM. P <0.05, P<0.001.

FIG. 8: Analysis of biochemical redox parameters on cortex, basal ganglia, cerebellum, pancreas, heart and skeletal muscles in YG8R mice injected Au₈-pX. (A) mitochondrial ATP levels are significantly increased in all tissues of Au₈-pX injected mice compared to control animals, except for cerebellum and pancreas. (B) the analysis of mitochondrial ROS revealed no statistically significant difference between injected and control animals, except for anterior tibial ganglia and basal ganglia. (D) mitochondrial lipid peroxidation (OH-nonenal) was reduced in Au₈-pX injected mice compared to control in all tissues tested except for the cortex and heart. (E) damage to mitochondrial DNA (8-oxo-guanine) was reduced in mice Au₈-pX injected compared to control in all tissues tested except for cortex, heart and pancreas (for DNA damage). (F) mitochondrial levels of SOD were significantly decreased in the cortex, cerebellum, quadriceps femoral, anterior tibial and soleo muscles of Au₈-pX injected mice. The values represent the mean±SEM.

FIG. 9: Analysis of biochemical redox parameters on cortex, basal ganglia, cerebellum, pancreas, heart and skeletal muscles in YG8R mice Au₈-pX injected. (A) GSH levels are increased in Au₈-pX injected mice in all tissues except the pancreas and heart. (B) GSSG levels are significantly decreased in mice injected Au8-PX into the cortex, basal ganglia, cerebellum and soleo. (C) GST levels are significantly increased in Au₈-pX injected mice in basal ganglia, cerebellum, femoral quadriceps, and anterior tibial.

FIG. 10: Image analysis (A) MSC proliferation derived from bone marrow FRDA or control subjects, exposed or not exposed to Au₈-pX, 5 or 10 μM. (B) production of ROS in MSCs derived from bone marrow FRDA or control subjects, exposed or not exposed to Au₈-pX 5 or 10 μM.

The present invention relates to superstructured gold clusters, called Au-pX, for use in the treatment of oxidative stress. By cluster is meant aggregates of atoms composed of metal atoms in a number comprised between 2 and 100, or which maintain dimensions lower than 2 nanometers, according to the definition provided by Yamazoe S et al. (Seiji Yamazoe, Kiichirou Koyasu, Tatsuya Tsukuda, Accounts of Chemical Research 2014 DOI: 10.1 021/ar400209a).

In a preferred embodiment, said superstructured gold clusters are obtained according to the method described in Santiago-Gonzalez et al. 2016, 353: 571-575. In a preferred embodiment, said Au-pX are obtained starting from clusters composed of 8 gold atoms (Au₈) and are an aggregate of 8 gold atoms. Said gold clusters are the basic components of a colloidal supramolecular superstructure called Au₈-pX (diameter 4-5 nanometers), bridged together by hydrogen bonds which form between the closure ligands.

In a preferred embodiment, said ligand is 11-mercaptoundecanoic acid which binds the gold atoms through the thiol groups exposing the carboxylic functionality.

In a preferred embodiment, the use of said Au-pX is in the treatment of Friedreich ataxia (FRDA). In a further embodiment, said use is in the treatment of Alzheimer's (AD), Parkinson's (PD) and/or Huntington's disease (HD).

In a preferred embodiment, said Au-pX superstructured gold clusters are included in a composition which also comprises pharmaceutically acceptable excipients for intravenous administration. A method of therapeutic treatment of subjects suffering from pathologies oxidative stress related, such as AD, PD, HD, is also described.

Said method comprises the intravenous administration of a composition comprising the Au-pX superstructure gold clusters according to the present invention. In a preferred embodiment, said method comprises a single administration.

The advantage of the method according to the present invention is the ability of Au-pX to interact with different types of cells within multiple tissues including nerves, skeletal muscles and cardiac systems by inducing long-term effects on mitochondrial activity. Although ROS levels remain unchanged, a reduction in oxidative stress and DNA damage with increased mitochondrial activity in nerve, skeletal and cardiac tissues of YG8R animals treated with Au₈-pX has been surprisingly demonstrated here. The Au-PX do not have a sacrificial role, so that their effect, after a single administration, is maintained over time. This allows to carry out a treatment of a subject suffering from an excess of oxygen radicals disease with a single administration.

The following examples have the sole purpose of better illustrating the invention and are in no way limiting the invention. The scope of the invention is defined by the following claims.

### EXAMPLE 1

### Synthesis of Colloidal Superstructures Based on Au8 Gold Clusters (Au₈-pX).

Au₈ cluster-based superstructures were obtained as described in Santiago-Gonzalez et al. Briefly, the following procedure was followed:

Synthesis of gold nanoparticles (AuNP): AuNP were obtained by adding 1 ml of 1M NaOH (Sigma Aldrich, pellet >98%, anhydrous) to 90 ml of ultrapure water (Chromasolv plus, HPLC grade). 2 ml of Tetrakis, solution of hydroxymethyl phosphonium chloride (THCP) (Sigma Aldrich) prepared by mixing 24 ml of THCP with 2 ml of water were then added. The mixture was stirred for 5 minutes, followed by the addition of 3 ml of 0.03 M HAuCl₄. 3H₂O (Sigma Aldrich, 99.999% traces of metal bases). The brown colour of the obtained solution indicates the reduction of Au⁺³ to Au° and the formation of gold particles of 2-3 nm.

Synthesis of Au₈ cluster superstructures (Au₈-pX): This nanomaterial is obtained by etching the previously synthesized AuNP. 2 ml sodium phosphate buffer 100 mM (pH 7) was added to 10 ml AuNP (stored at 4° C.). 2 ml 11-mercaptoundecanoic acid (MUA) 0.1 M containing an equivalent amount of NaOH (0.2 ml of 1M NaOH added to 2 ml of water and MUA dispersion) are then added. The pH of the solution is adjusted to pH=7.5 with 100 mm phosphate buffer at pH 2.5 and pH 9. The mixture was protected from light and allowed to react for 72 hours in a refrigerator. The resulting pale yellow solutions were purified several times by centrifugation at 11000 g for 30 minutes to remove excess thiols and filtered through a Whatman syringe membrane filter (0.22 μm pore size) to remove any remaining particle aggregates from the solution. The product obtained is Au₈-pX.

### EXAMPLE 2

### Measurement of the Catalyzing Effect Exerted by the Au₈-pX Over-Structured Gold Clusters on the Dissociation of Hydrogen Peroxide in an Aqueous Medium

The purpose of the experiment was to verify electrochemically, by means of cyclic voltammetry, the non-sacrificial catalytic activity of the clusters. The measurements were carried out in a context as close as possible to the biological one, using PBS as electrolyte, a saline phosphate buffer solution commonly used to reproduce the cell environment, and introducing into the system a controlled aliquot of hydrogen peroxide (H₂O₂) and/or Au₈-pXs clusters in concentration 345 μM.

Electrochemical measurements were carried out in a three-electrode cell with the following characteristics:

Working electrode (WE): Selected from gold pin electrodes, Glassy carbon and FTO glass pin, i.e. glass on which a conductive and transparent oxide layer is deposited, on which thin films of Au₈-pXs have been deposited by drop casting.

Reference electrode (RE): For measurements in an aqueous medium, the saturated calomel electrode (SCE) was used while for the organic environment a pseudo-reference of Ag/AgCl was used, then calibrated with ferrocene.

Counter electrode (CE): For measurements in aqueous medium, Glassy carbon pin was used while in organic medium a platinum mesh electrode was used.

All measurements were carried out using PARSTAT 2273 potentiostat/galvanostat (Princeton Applied Research). The starting electrolyte volume used is 3 ml.

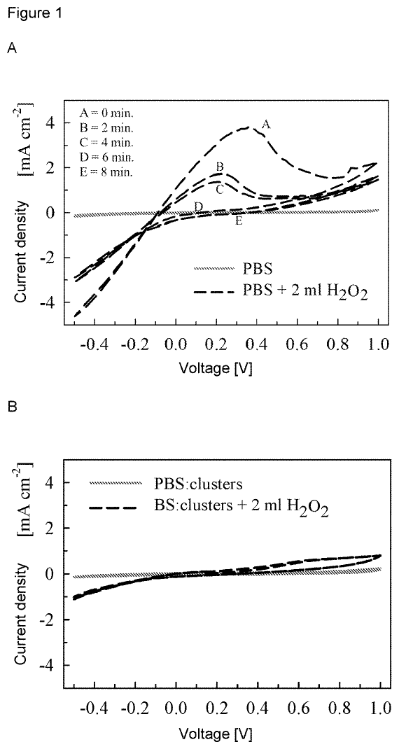

The results obtained are reported in FIG. 1. Panel A shows the cyclovoltagram of the PBS electrolyte before (solid line) and after (dashed line) the addition of 2 ml of H₂O₂ aqueous solution (0.3% by volume).

While the PBS proves to be totally inactive, the peak current due to oxidation of the hydrogen peroxide is evident for voltages higher than 0.1 V. Over time, the signal strength decreases until it disappears when all the peroxide added to the solution has been oxidized. Panel B shows the cyclovoltagram of the PBS electrolyte containing clusters Au₈-pXs 345 μM before (solid line) and after (dashed line) the addition of 2 ml of H₂O₂ aqueous solution (0.3% by volume). The solid line which, although in the presence of Au₈-pXs, does not undergo any modification with respect to what was observed in the presence of PBS alone, is indicative of the fact that the clusters are inactive, presenting no current peak due to any oxidation or reduction reaction. Conversely, the dashed line is deeply modified from panel A to panel B, where, in the presence of Au₈-pXs clusters, the current peak relative to the oxidation of H₂O₂ completely disappears. The data is indicative of the role of Au₈-pX as a catalyst in the dissociation reaction of hydrogen peroxide, which is therefore no longer available for the oxidation reaction.

### EXAMPLE 3

### Measurement of the Scavenging Effect of the Au₈-pXs Clusters on Different Reactive Oxygen Species (ROS)

Singlet Oxygen

The effect of the presence of clusters on singlet oxygen ¹O₂ was measured with photoluminescence techniques in solution, using the commercial fluorescent sensor Singlet Oxygen Sensor Green (SOSG, Invitrogen™). SOSG is a conjugated organic molecule which, optically excited in the presence of ¹O₂, undergoes a photo-oxidation reaction which makes it luminescent. The intensity of the photoluminescence signal of the SOSG is therefore proportional to the concentration of ¹O₂ dispersed in solution. In the context of the example, ¹O₂ is produced in a controlled manner using Rose Bengal (RB) as the sensitizer photo.

100 μg of SOSG are dissolved in 1 ml of methanol. The solution was diluted 1:5 in HPLC water and divided into two samples. To one of these, RB 10⁻⁵ M was added The gold clusters are prepared in an aqueous solution at the concentration 345 μM.

The measurements are carried out on 4 samples prepared as follow:

−1 ml of aqueous solution RB:SOSG+1 ml of H₂O.

−1 ml of aqueous solution RB:SOSG+1 ml of solution of Au₈-pXs.

−1 ml of SOSG+1 ml of H₂O aqueous solution.

−1 ml of SOSG aqueous solution+1 ml of Au₈-pX solution.

To generate ¹O₂, the RB is excited with a non-focused 532 nm CW laser with a power of 0.3 mW. The SOSG detector is energized with a 473 nm non-focused CW laser with a power of 0.3 mW. The intensity of the photoluminescence signal of the SOSG, proportional to the concentration of ¹O₂ in solution, is recorded with a CCD Spec 2000 detector (Horiba Jobin-Yvon) coupled to a Triax 190 monochromator.

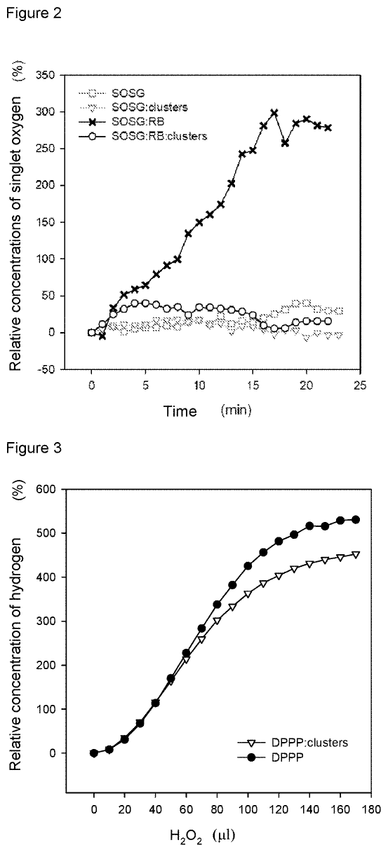

The results, shown in FIG. 2, show the percentage variation in the concentration of ¹O₂ in solution as a function of time in the set of samples prepared. No significant increase is recorded in samples without RB (gray lines). In samples containing RB (black lines), the amount of ¹O₂ increases by 300% over the 25 minutes in which the measurements were made (line marked with crosses). The presence of gold clusters (line marked with circles) significantly reduces the ¹O₂ concentration showing a final increase of only 15%, which is fully comparable to that observed in samples without RB. The data show that the cluster scavenging effect versus ¹O₂ reduces the final concentration of ROS in the sample by a factor of 20.

Hydrogen Peroxide

The effect of the presence of clusters on H₂O₂ hydrogen peroxide was measured by photoluminescence techniques in solution using diphenyl-1-pyrenylphosphine (DPPA, Invitrogen™) as the luminescent sensor. DPPP is a phosphine that has no luminescence, until oxidized by interaction with H₂O₂. In this form, DPPP exhibits an optical absorption peak in the near UV and an emission at 380 nm. The intensity of the luminescence of the DPP is therefore proportional to the concentration of H₂O₂ in solution.

DPPP is dissolved in ethanol solution at a concentration of 10⁻⁴ M. The hydrogen peroxide solution used in the experiment was prepared by diluting a 30% by volume hydrogen peroxide solution by a factor of 1:100.

The gold clusters are prepared in an aqueous solution at a concentration of 345 μM.

The measurements were carried out on two samples formulated as follow:

−1.5 ml of DPPPP solution+0.25 ml of _{H202}.

−1.5 ml of DPPA solution+0.25 ml of Au₈-pX solution.

The measurement was carried out by monitoring the intensity of the photoluminescence of the DPP as a function of the amount of H₂O₂ added to the starting solution, under continuous illumination with an unfocused 355 nm laser with a power of 3 mW. The photoluminescence signal of DPPP was recorded with a CCD Spec 2000 detector (Horiba Jobin-Yvon) coupled to a Triax 190 monochromator.

The results, shown in FIG. 3, show that, in the absence of gold clusters (circles), the addition of 170 ml of H₂O₂ results in an increase in ROS concentration of 530%. The presence of gold clusters (triangles) does not significantly affect the final concentration of H₂O₂, which shows a relative variation of 440%. The data is indicative of the fact that the digesting activity toward the hydrogen peroxide of the gold clusters, although present as shown also in example 2, is much lower than the digesting activity toward the singlet oxygen.

Radical Oxygen

The effect of the presence of gold clusters on radical oxygen O_{\dot{2}}⁻ was measured by photoluminescence techniques in solution, using MITOSOX™ red (Invitrogen™) as a commercial luminescent sensor. MITOSOX™ red is a molecule that becomes luminescent selectively upon reaction with the superoxide radical. The intensity of the luminescence of the MITOSOX™ red is therefore proportional to the concentration of O_{\dot{2}}⁻ dispersed in solution. In this case, ROS is generated by exploiting the photolysis of hydrogen peroxide as a generator of this species, as described in Environ. Skiing. Technol., you. 41, no. 21, pp. 7486-7490, 2007).

50 μg of MITOSOX™ red are dissolved in 0.5 ml of DMSO, then adding 4.5 ml of H₂O₂. To generate radical oxygen, H₂O₂ was added 10 μl dropwise from a 3% volume aqueous solution. The gold clusters are prepared in an aqueous solution at a concentration of 345 μM.

The measurements were carried out on two samples formulated as follow:

−1 ml of a MITOSOX™ red+0.5 ml of H₂O₂ solution.

−1 ml of MITOSOX™ red solution+0.5 ml of Au₈-pX solution.

The measurement was carried out by monitoring the intensity of the photoluminescence of the MITOSOX™ red as a function of the amount of H₂O₂ added to the starting solution, under continuous illumination with a laser at 405 nm not focused at a power of 23.5 mW. The source used is able simultaneously to activate both photolysis of the hydrogen peroxide necessary to generate O_{\dot{2}}⁻ and the luminescence of MITOSOX™ red activated by the interaction with the O_{\dot{2}}⁻ molecules photogenerated. The intensity of the photoluminescence signal was recorded with a CCD Spec2000 detector (Horiba Jobin-Yvon) coupled to a Triax 190 monochromator.

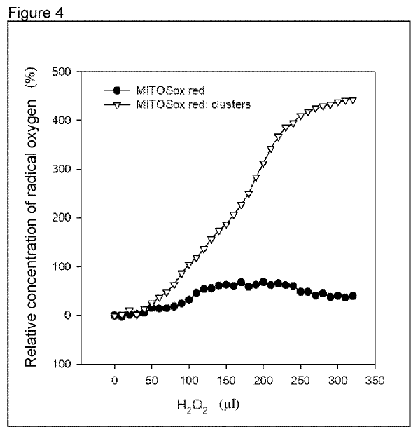

The results, shown in FIG. 4, show the percentage variation of the concentration of radical oxygen O_{\dot{2}}⁻ as a function of the amount of the photo sensitizer H₂O2 added to the starting solution, under illumination at 405 nm. In the gold cluster-free sample (black circles), the addition of 300 μl of H₂O₂ results in an increase in ROS concentration of 440%. The presence of clusters of gold (triangles) significantly reduces the increase of O_{\dot{2}}⁻, which shows a final relative variation of 45%. In the case of radical oxygen, the cluster scavenging effect reduces the final concentration of ROS in the sample by a factor of 10.

### EXAMPLE 4

### In Vitro Evaluation of Proliferation and Production of ROS in Mesenchymal Stem Cells Derived from the Bone Marrow of FRDA Patients

Isolation of mesenchymal stem cell (MSC) cells derived from the bone marrow of the FRDA patient.

Samples were obtained from 3 FRDA subjects, after collecting the informed consent. 6 ml of bone marrow were aseptically aspirated from the left posterior iliac crest with local anaesthesia in a sterile manner. The collected bone marrow was filtered through a cell filter (100 μm) to remove any bone spicles or clots present. The method of lysis of red blood cells was used for the extraction of MSC. The collected sample was transferred to a 50 ml conical centrifuge tube and was added, in the 1:5 (v/v), the erythrocyte lysis buffer, ACK solution (NH₄Cl 150 mM, KHCO₃ 10 mM and Na₂EDTA0.1 mM) the tube was stirred manually for 1 minute and then centrifuged for 5 minutes at 480 g. The precipitated bone marrow was then diluted with the respective culture media in a ratio of 1:1. The mononuclear cell fraction (MNC) from the bone marrow was separated from the gradient density centrifugation with Lymphoprep™ (1.077 g/ml). 2.5 ml of Lymphoprep™ are collected in a sterile 15 ml centrifuge tube and stratified with 5 ml of diluted bone marrow (ratio 1:2) without mixing it with the Lymphoprep layer. The sample was then centrifuged at 1800 rpm for 20 minutes at room temperature (RT). The MNC accumulated in the plasma prep interphase of the lymph, in the buffy coat layer, was carefully isolated by suction, transferred to a new 15 ml centrifuge tube and suspended in culture media. The total volume of resuspended pellet was transferred to a 175 cm² ventilated flask and cultured for 24 hours in DMEM medium containing 10% FBS in the incubator under standard conditions 5% CO₂, 37° C. After 24 hours, the medium was removed and the cells were washed with phosphate buffered saline (PBS) to remove the non-adherent cells. MSC Basal Medium (DMEM/F12, 1:1) (Thermo Fisher Scientific-US) containing 10% FBS (Thermo Fisher Scientific-US) was used for the subsequent cultivation of MSCs. The medium was completely changed every 3-4 days. When the adherent cells became confluent, the MSCs were treated with trypsin-EDTA (Invitrogen, UK), washed twice with PBS, counted and distributed in new 175 cm² flasks at a density of 2×10⁶ cells/flask, incubated in the incubator under standard conditions (5% CO₂, 37° C.).

Image Analysis

Bone marrow mesenchymal stem cells (BM-MSC), derived from either a healthy donor (ctr) or a patient (ftx), were seeded at the concentration of 75000 cells/cm² in a 24-well plate, in order to have three experimental replicas for each tested condition. After 24 hours from seeding, at 70% of cell confluence, three wells with BM-MSC ctr and three with BM-MSC ftx were treated with 5 and 10 μM Au₈-pXs. The images were acquired with the IncuCyte Vive Cell Analysis System (Sartorius). The experiment lasted 24 hours and four pictures for each well were taken with a 10× objective every 4 hours. The results were analyzed with the IncuByte software (Sartorius), setting the instrument to create the cell mask that best adapted to all the different conditions tested and the area of the cell per well was correlated with time.

The results obtained, shown in graph A of FIG. 10, indicate that MSCs derived from bone marrow FRDA (MSC ftx) exhibit markedly higher proliferation in the presence of Au₈-pXs 5 or 10 μM as compared to the proliferation in the absence of the treatment. The degree of proliferation achieved is comparable to that observed in MSC ctr.

ROS tests

For the evaluation of reactive oxygen species (ROS) possibly produced in culture, the cells were seeded as for the image analysis test. The analysis was carried out 24 hours after the addition of Au₈-pX in the culture medium. ROS-Glo TM H₂O₂ (Promega) assay was used, following the manufacturer's protocol. The non-lithic assay was performed and the relative luminescence units were measured by a plate reader (GloMax

discover, Promega).

The results obtained, shown in graph B of FIG. 10, indicate for MSC ftx exposed to Au₈-pX 5 or 10 μM a lower ROS production than observed in untreated MSC ftx.

The results are indicative of the ability of Au₈-pX to lower ROS levels and consequently to limit their cytotoxicity without having their intrinsic cytotoxicity.

### EXAMPLE 5

### Evaluation of the Effect of AU8-PX in a FRDA Mouse Model

Animal Model

Frataxin YG8R mice (Jackson Laboratory # 024113) are an accepted murine model of Friedreich ataxia (Anjomani Virmouni et al., 2015 Mol Neurodegener. 10:22). In particular, Frataxin YG8sR mice begin to show signs of cardiomyopathy and motor deficiency at the age of 9 months, also show glucose tolerance and insulin resistance defects and histological signs of cell damage in the brain, muscles, DRG. The animals appear generally normal, they can feed themselves, but they are difficult to reproduce. The animals were maintained in a mixed genetic background C57BL6/J.

Preparation of Au₈-pX and Injection

For each experimental group, ten 12-month Frataxin YG8R mice were evaluated (5 males and 5 females). All animals received an Au₈-pX intravenous (IV) injection in the tail vein using a syringe with a thin needle (avoiding deposit of clusters). Based on in vitro evidence, a therapeutic dose of 10 μM of Au₈-pX was estimated, corresponding to 300 μg of clusters per mouse of 20 g weight. To avoid aggregation of clusters in vivo and lung damage during intravenous injections, animals received a dose of 100 μg of Au₈-pX suspended in 100 μl saline per week for three weeks.

Motor test

Footprints: To obtain fingerprints, the mouse legs were immersed in a non-toxic water-based food dye. Mice were allowed to walk along a walkway 40 cm long and 9.5 cm wide (with side walls 7 cm high) with white paper covering the floor. All mice had a training run and were then subjected to three tests. Three steps of the center portion of each run were measured with a total number of nine steps per mouse for the length of the posterior pitch and the anterior left pitch, the length of the posterior and anterior right pitch, the width of the anterior base (the width between the right and left front limbs) and the width of the rear base (the width between the right and left rear limbs).

Treadmill: The resistance to exercise was tested with the treadmill. Mice were placed on a transparent treadmill belt (CleverySys Inc), with a constant slope of 10% and a gradual increasing rotational speed. The following program was used: Speed 18 cm/sec, 0 to 10 minutes; speed 28 cm/sec, 10 to 20 min, speed 38 cm/sec, 20 to 25 min, speed 42 cm/sec, 25 to 30 min. Mice were trained for three weekly sessions prior to registration. The data collection is started at a rate of 38 cm/sec. At each time point, the number of accumulated errors is recorded; in case of apparent physical exhaustion before the end of the test, the animals are removed from the apparatus and an arbitrary value is assigned based on the total distance traveled. The tests were repeated at weekly intervals.

Echocardiography

Transthoracic echocardiography was performed with a small, high-resolution imaging system for animals (VeVo2100, VisualSonics, Inc, Toronto, Canada) equipped with a 22-55 MHz transducer (MicroScan transducers, MS500D). Mice, anesthetized by inhalation of iso-uranus (2%) and maintained by mask ventilation (iso-uranus 1%), were placed in a low left side decubitus position, with strict thermoregulation (37±1° C.) to optimize physiological conditions and reduce hemodynamic variability. The fur was removed from the chest by applying a cosmetic cream to obtain a clear image. The echocardiographic parameters were measured at the papillary muscles in the short axis parasternal view (M mode). Fractional shortening LV was calculated as follows: FS=((LVEDD−LVESD)/LVEDD)×100, where LVFS indicates fractional shortening LV; LVEDD, end diastolic diameter LV; and LVESD, end systolic diameter LV. The LV ejection fraction was automatically calculated by the echocardiographic system. All measurements were averaged over 5 consecutive cardiac cycles per experiment and cardiac function was evaluated when heart rate as 450 to 500 bpm.

Real Time qPCR

For the quantitative analysis of mRNA expression, the tissue fragments isolated and sectioned after injection and from control animals (not injected) at the time of sacrifice were immediately immersed in the Trizol reagent (Roche) and extracted as indicated by the manufacturer. RNA quality, primer efficiency and correct product size were verified by RT-PCR and agarose gel electrophoresis. Real time qPCR was performed with LightCycler (Roche) using FastStart DNA MasterPLUS SYBR-Green I (Roche). 2 μl of cDNA were used in each reaction. All samples were tested in triplicate. The specificity and absence of primer dimers was controlled by denaturation curves; for each mRNA examined, only one denaturation peak was observed. Glyceraldehyde-3-phosphate dehydrogenase (GAPDH) was used for normalization, calculated using LightCycler software 3.5.3.

Biochemical Analysis of Cellular Metabolism

Tissues were sectioned and immediately frozen by immersion in liquid nitrogen, then pulverized and stored at −70° C. To isolate the mitochondrial fractions, the tissue powders were washed twice in ice cold PBS, lysed in 0.5 ml of mitochondria lysis buffer (50 mmol/l Tris, 100 mmol/l KCl, 5 mmol/l MgCl₂, 1.8 mmol/L ATP, 1 mmol/L EDTA, pH 7.2), supplemented with protease inhibitor cocktail III (Calbiochem, La Jolla, Calif., USA), 1 mmol/l PMSF and 250 mmol/l NaF. The samples were clarified by centrifugation at 650 g for 3 minutes at +4° C.: the supernatant was collected and centrifuged at 13000 g for 5 minutes at +4° C. The pellet—containing the mitochondria—was washed once with lysis buffer and resuspended in 0.25 ml of a resuspension buffer composed of 250 mmol/l sucrose, 15 mmol/L K₂HPO₄, 2 mmol/L MgCl₂, 0.5 mmol/L EDTA. A 50 μl aliquot was sonicated and used for the measurement of protein content or western blot. To confirm the presence of mitochondrial proteins in the extracts, 10 μg of each sonic sample were SDS-PAGE submitted and probed with an anti-porin antibody (Abcam, Cambridge, United Kingdom).

The amount of ROS in whole cells or mitochondria extracts was measured by labelling the samples with the ROS 5- (E-6)-chloromethyl-2′,7′-dichlorodihydro-fluorescein diacetate-acetoxymethyl ester (DCFDA-AM)-sensitive fluorescent probe. The results were expressed as nmol/mg of cellular proteins or mitochondrial proteins.

To measure the flow of electrons from complex Ito complex III, taken as an index of mitochondrial respiratory activity, 50 μg of non-sonic mitochondrial samples were resuspended in 0.2 ml of Buffer A (5 mmol/l KH₂PO₄, 5 mmol/l MgCl₂, 5% w/v BSA) and transferred to a quartz spectrophotometer cuvette. Then 0.1 ml of buffer B (25% w/v of saponin, 50 mmol/L KH₂PO₄, 5 mmol/L MgCl₂, 5% w/v BSA, 0.12 mmol/L c-oxidized form of cytochrome, 0.2 mmol/L NaN₃) added for 5 minutes at room temperature. The reaction started with 0.15 mmol/l of NADH and was followed for 5 minutes, reading the absorbance at 550 nm with a Packard EL340 microplate reader (Bio-Tek Instruments, Winoski, Vt., USA). The results were expressed as nanomoles of reduced cytochrome c mitochondrial proteins/min/mg. The amount of ATP in mitochondrial extracts was measured with the bioluminescent ATP assay kit (Sigma-Aldrich). The results were expressed in nmol/mg of mitochondrial proteins.

The amount of ATP produced by oxidative phosphorylation was measured on 20 μg of mitochondrial proteins with the ATP Bioluminescent Assay Kit (FL-AA; Sigma Chemical Co.). The data were converted to nmol/mg of mitochondrial proteins, using a previously set calibration curve. The amount of oxidative damage was measured in total tissue extracts and mitochondrial extracts by two independent assays: 1) quantitative measurement of lipid peroxidation (OH-nonenal) ELISA (Abcam, Cancridge, United Kingdom), the results are expressed in nmol/mg of cellular protein or/mg of mitochondrial protein; 2) quantitative measurement of 8-oxo-deoxy-guanine (DNA damage) by ELISA (Abcam Cambridge, United Kingdom), the results are expressed in nmol/μg of DNA.

To measure SOD1 and SOD2 activity, the mitochondria were isolated as previously reported (Riganti et al., 2013). The activity of cytosolic SOD1 and mitochondrial SOD2 was measured using 10 pg of each extract, incubated with 50 μmol/L xanthine, 5 U/mL xanthine oxidase, 1 μg/mL cytochrome oxidized C.

The rate of reduction of cytochrome c, which is inhibited by the presence of SOD, was monitored for 5 minutes by reading the absorbance at 550 nm with a Packard EL340 microplate reader (Bio-Tek Instruments, Winooski, Mont.). The results were expressed as reduced cytochrome C μmol/min/mg cytosolic or mitochondrial proteins.

The total, reduced glutathione (GSH) and oxidized glutathione (GSSG) content was measured by a colorimetric method, using a Packard EL340 microplate reader (Bio-Tek Instruments), as described in detail (Riganti etal., 2006). The results were expressed as pmol of glutathione/mg of cellular proteins. For each sample, GSH was obtained by subtracting GSSG from the total glutathione. GST activity was measured using the glutathione S-transferase (GST) test kit (Sigma Chemicals. Co), according to the manufacturer's instructions. The results were expressed in pmol of CDNB-GSH adducts/min/mg of proteins.

Lipoperoxidation: 100 μg of whole tissue homogenate proteins and 50 μg of extracted mitochondria proteins (Riganti et al., 2013) were tested with the lipid peroxidation kit (4-HNE), to evaluate the amount of 4-hydroxynonenal (4-HNE), an oxidation index of proteins. The results were expressed in nmol/mg of total or mitochondrial proteins.

DNA damage: 50 ng of DNA extracted from whole tissue homogenate and 10 ng of mitochondrial DNA, extracted from isolated mitochondria (Riganti et al., 2013), were evaluated with the 8-Hydroxy-2′-deoxyguanosine ELISA kit (Abcam, Cambridge, United Kingdom), to detect oxidative damage to DNA. The results were expressed in nmol/μg of mitochondrial or total DNA, respectively.

Results:

Treatment with Au₈-pX improves neuromotor and cardiac functions of elderly YG8R mice.

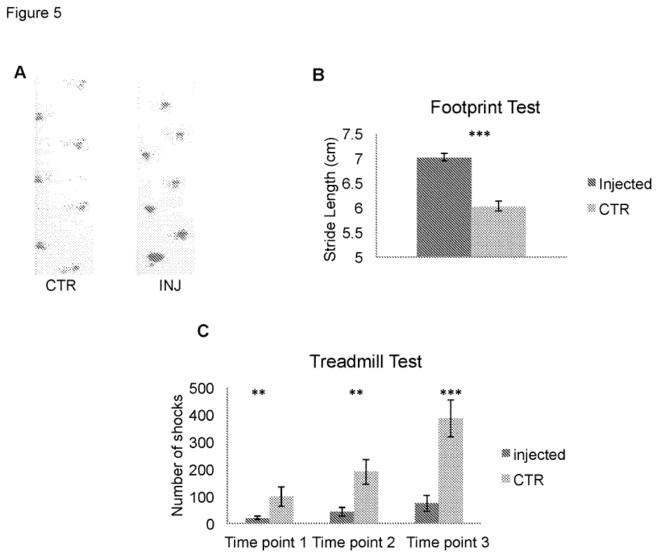

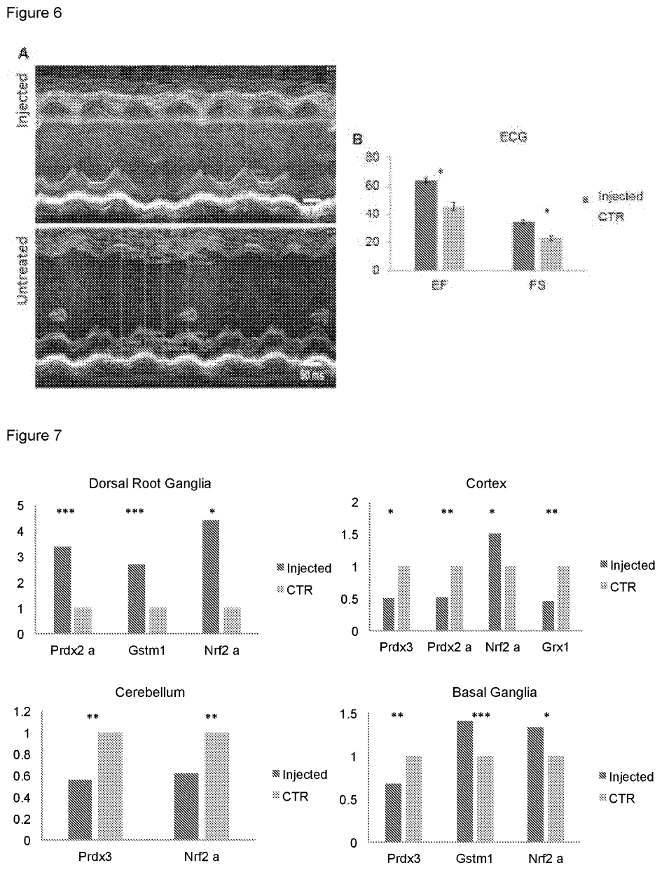

Overall, the motor capacity was evaluated in terms of motor coordination with foot print tests and exhaustion time and resistance time with treadmill performance in untreated elderly YG8R mice (n=10; 5 females and 5 males) and treated (n=10; 5 females and 5 males). YG8R mice treated with Au₈-pX (n=10) were injected when clinically symptomatic for coordination and motor deficiency (12 months of age) and tested from 2 months of age (asymptomatic) to six months after injection until sacrifice (18 months of age). YG8R mice showed a progressive reduction in locomotor activity and coordination deficiency as previously described (Al-Mandawi S, et al. Genomics.2006; 88: 580-590; Virmouni Anjoli S, et al. DIS Model Mech.2015; 8: 225-235). There was a clear improvement in footprint tests for motor coordination in treated YG8R mice compared to untreated mice (FIG. 5A, B). In particular, the resistance time, measured at time point 1, 2 and 3, was improved in older YG8R mice treated with Au₈-pX with an increase of ˜40% compared to the resistance time of untreated YG8R animals (FIG. 5C). Echocardiographic analysis in YG8R mice of 18 months, the results of which are shown in FIG. 6, revealed a decrease in end LV systolic/diastolic volumes (n=10) with a significant improvement in the left ventricular ejection fraction (LV) after the Au₈-pX treatment (n=10). (Systolic end LV volume: p=0.0123; end diastolic volume LV: p=0.0362; ejection fraction: p=0.0130). In addition, significant downregulation was observed in the volume of the scale stroke (SV) (p=0.0049), in the left ventricular inner diameter in diastole (LVID; p=0.0148) and in the length of the diameter in systole (diameter; s) and diastole (diameter; d) (respectively, p=0.0309 and p=0.0394).

Au₈-pX affects the redox pathways of YG8R mice.

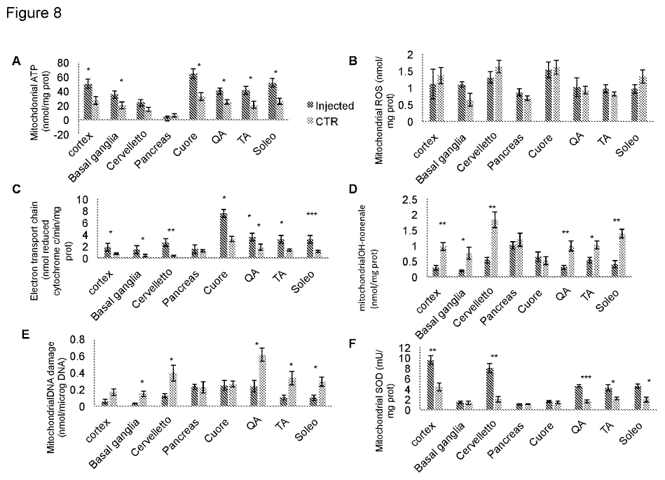

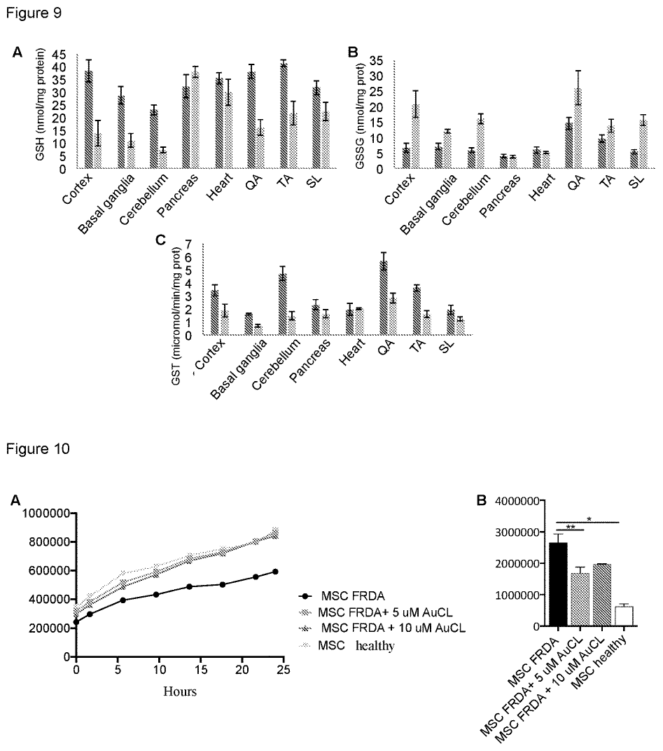

The accumulation of oxidized proteins and mitochondrial dysfunction were previously documented in YG8R mice (Shan Y., et al. 2013 10.1089/ars.2012.4537; Celine J. Rocca, et al. Ski Transl Med. 2017 9:413). Significant changes in expression of Peroxiredoxin, Glutaredoxin, Glutathione-S-transferase and Nrf2 were found in the brain, spinal cord, DRG (FIG. 7), pancreas and muscle tissues of YG8R treated with Au₈-pX. No difference in ROS levels, both in total tissue extracts and in isolated mitochondria was found between untreated and treated YG8R mice (data not shown). In nerve muscle tissue samples (cortex, cerebellum, base ganglia) and skeletal (TA, VM and soleus) of the Au₈-pX injected mice there was a reduction in the lipid peroxidation of cytosol and mitochondria (OH-nonenal, FIG. 8D) and in the ROS-dependant DNA injury (deoxy-guanine bone, FIG. 8E), while an increase in SOD2 and GSH levels and a decrease in GSSG levels were observed, accompanied by increased GST activity (FIG. 9). In all these tissues and in the heart, the mitochondrial electron transport chain (FIG. 8C) and the mitochondrial ATP level (FIG. 8A) were significantly increased in Au₈-pX treated compared to untreated YG8R. Together, these data showed that the injection of Au₈-pX reduced oxidative damage and improved mitochondrial function in YG8R mice, consistent with neuromotor and functional cardiac improvement.

1. Superstructured gold clusters Au-pX, which consist of gold atoms and at least one ligand, where said gold atoms in said cluster are in a number comprised between 2 and 100 or the cluster dimension are smaller than 2 nm, for use in the treatment of diseases related to oxidative stress.

2. Superstructured gold clusters Au-pX for use according to claim 1, where said gold atoms are 8, and said superstructured cluster is called Au8-pX.

3. Superstructured gold clusters Au-pX for use according to claim 1, where said ligand is 11-mercaptoundecanoic acid.

4. Superstructured gold clusters Au-pX for use according to claim 1, where said pathology is determined by excess ROS.

5. Superstructured gold clusters Au-pX for use according to claim 1, where said pathology is selected from Friedreich's ataxia (FRDA), Alzheimer's disease (AD), Huntington disease (HD), Parkinson's disease (PD).

6. Superstructured gold clusters Au-pX for use according to claim 1, where said pathology is Friedreich's ataxia (FRDA).

7. Superstructured gold clusters Au-pX for use according to claim 1, where said treatment is intravenously.

8. Superstructured gold clusters Au-pX for use according to claim 1, wherein said treatment consists of a single intravenous administration.

9. Composition comprising superstructured gold clusters Au-pX, which consist of 8 gold atoms and at least one ligand and pharmacologically acceptable excipients for intravenous administration.
